(19) United States
(12) Patent Application Publication
(10) Pub. No.: US 2025/0352688 A1
(43) Pub. Date: Nov. 20, 2025

(54) AIR PURIFIER FOR INTERNAL SPACES

(71) Applicant: Ecological Balancing Technologies Corporation, Wilmington, DE (US)

(72) Inventor: Michael HOFFMAN, Moshav Udim (IL)

(73) Assignee: Ecological Balancing Technologies Corporation, Wilmington, DE (US)

(21) Appl. No.: 18/866,087

(22) PCT Filed: May 17, 2023
(86) PCT No.: PCT/IL2023/050512

§ 371 Date: Nov. 15, 2024

Related U.S. Application Data
(60) Provisional application No. 63/342,639, filed on May 17, 2022.

(51) Int. Cl. A61L 9/14; A61L 9/20; F24F 8/108; F24F 8/158

(52) U.S. Cl. CPC A61L 9/14; A61L 9/20; F24F 8/108; F24F 8/158; A61L 2209/111; A61L 2209/133; A61L 2209/14; A61L 2209/16

(57) ABSTRACT

Air purification device for internal spaces comprises an air inlet to receive air from an internal space, a filter to filter air from the air inlet; a probiotic dispersion system to insert probiotic material into the air after the filter; and an outlet through which to distribute the air into the internal space.

### RELATED APPLICATION/S

This application claims the benefit of priority of U.S. Provisional Patent Application No. 63/342,639 filed on 17 May 2022, the contents of which are incorporated herein by reference in their entirety.

### FIELD AND BACKGROUND OF THE INVENTION

The present invention, in some embodiments thereof, relates to an air purifier and, more particularly, but not exclusively, to an air purifier for an indoor setting.

According to the World Health Organization and the Center for Disease Control, our indoor space is substantially more polluted than the outdoors. And, the indoor pollution is mostly concentrated on surfaces and objects, reaching up to a concentration of 5× higher than in the air. Due to gravity, pollution in the air rapidly descends onto the surfaces below, where mold and bacteria continue to depend on the contaminated objects as their food source since food is only consumed when resting on solid objects—ultimately, contact with surfaces and objects may cause 80% of infectious disease transmissions.

None of the air purifiers in the market today, offer a safe and efficient solution; The method used by current filtering devices is to suck air into a cleaning chamber where it is cleaned by UV light beams or a physical barrier. There may be an active filter that is supposed to let air pass and only block the particles the air carries. However, none of these technologies make any attempt to clean the surfaces and objects; therefore, the purification of indoor spaces with air purifiers is mediocre at best and inadequate since the majority of bacteria, mold, viruses and allergens remain on the surfaces and are not removed. The surfaces thus continue to be sources of bad odors and of materials that trigger allergies and asthma.

### SUMMARY OF THE INVENTION

The present embodiments may combine air filtration with probiotic dispensing, so that helpful bacteria that produce friendly odors, or at least replace the harmful bacteria, fungi etc are encouraged to grow on the available surfaces while the air is cleaned.

In an embodiment, suction into the filter is suspended during probiotic dispensing.

According to an aspect of some embodiments of the present invention there is provided an air purification device for internal spaces comprising:
an air inlet configured to receive air from an internal space;
a filter configured to filter air from said air inlet;
a probiotic dispersion system configured to insert probiotic material into said air after said filter; and
an outlet configured to distribute said air into said internal space.

Embodiments may include one or more sensors to detect levels of air contamination in said internal space.

The sensors may include any of a particle sensor, a biological sensor, a humidity sensor and/or a temperature sensor, and may also include a micron-scale filter and an active carbon filter.

Embodiments may vary a rate of air filtration based on measurement results of said at least one sensor.

Embodiments may vary a rate of said insertion of probiotic material based on measurement results of said at least one sensor.

In embodiments, the filter may close during said insertion of probiotic material so that the probiotic material is not immediately removed following insertion.

The filtering process may include passing air through UV-C light, so as to sterilize any biologically active material in the air. Accordingly bacteria, fungi and viruses may be neutralized.

According to a second aspect of the present invention there is provided an air purification method for internal spaces comprising:
receiving air from an internal space;
filtering said received air;
inserting probiotic material into said air after said filtering; and
distributing said air into said internal space.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1C are three different views of an integrated air purification and air and surface treatment device according to embodiments of the present invention;

FIG. 2 is a simplified cross-sectional view from above of the device of FIG. 1A;

FIG. 3 is a simplified transverse cross-section of the device of FIG. 1A;

FIG. 4 is a simplified functional block diagram of the device of FIG. 1A;

FIG. 5 is a simplified block diagram of an embodiment of the present invention provided as a bypass to a built-in HVAC system;

FIG. 6 is a simplified diagram showing a unit that is placed in parallel with an HVAC system according to the embodiment of FIG. 5;

FIG. 7 is a simplified cross-sectional diagram of the unit of FIG. 6;

FIG. 8 is a simplified diagram of a refill bottle that provides microbiome material for either the standalone or HVAC embodiments of the present invention;

FIG. 9 is a simplified diagram showing the refill bottle with a dispensing head; and

FIG. 10 is a simplified diagram showing placement of the refill bottle within the standalone unit of FIG. 1A.

### DESCRIPTION OF SPECIFIC EMBODIMENTS OF THE INVENTION

The present invention, in some embodiments thereof, relates to an air purifier and, more particularly, but not exclusively, to an air purifier for indoor settings which also includes probiotic spray and thus integrates air purification and surface microbiome regeneration, removing general pollutants from the air as well as bacteria, viruses and allergens.

An air purifier according to the present embodiments may purify the air in an internal space from carrying infectious diseases, pandemic-causing viruses, antibiotic resistant bacteria, mold, pollen, pet dander or even bad odors. The present embodiments may combine air filtration with probiotic dispensing protection of the internal space. Probiotic dispensing applies friendly microorganisms to the surfaces in the room, thus integrating cleaning of the air with cleaning of the surfaces. The integrated action of the purifier removes bacteria and viruses as well as allergens from the air and replaces the removed microbiological agents with seeding for a healthier more beneficial microbiome.

The present embodiments may accordingly provide a dual-function integrated bio-cleaning device that balances the room's microbiome by dispensing probiotics technology, while thoroughly cleaning the air at the microscopic level, that is to say to the level of less than one micron, by deployment of suitable filtering technology, thus removing bacteria, viruses and allergens.

The probiotic technology may include ingredients that are natural, organic and safe to seed an improved microbiome for the room environment.

Embodiments may thus at the same time filter the air of impurities, deplete the pathogens in the air, and the resources on which they live, and replace the bacterial community on any given surface. The cleaning process may involve passive technology that is to say a filter through which air is drawn by suction and thus cleaned. A further aspect of the cleaning process may be proactive, involving dispersing beneficial micro-organisms that provide continuous cleaning of surfaces and objects.

In embodiments, a sensor-based algorithm may control the power level of the respective operations.

Thus, in the case of the air filter, when a sensor reports a higher concentration of particles in the air, the extent of filtering may accordingly be increased. Thus the air suction increases so as to filter more air in limited time. Similarly, more dispersion of probiotics may take place where sensors report higher concentrations of pathogens or allergens.

An algorithm may provides for smart co-existence of the two functions. Thus while probiotics are dispersed, suction of the air may be paused. Thus the probiotics are not immediately sucked into the filter and cleaned away. Accordingly the probiotics are left to spread around the room and settle on available surfaces.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

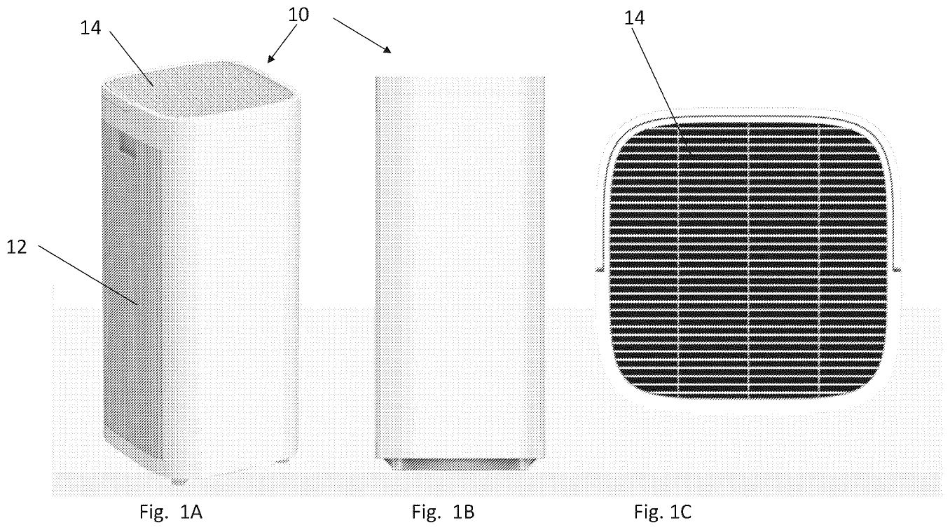

Referring now to the drawings, FIGS. 1A to 1C illustrate three views of an air purification device for internal spaces according to the present embodiments.

Air purification device 10 comprises an air inlet 12 that receives air from an internal space. For example air may be sucked in using a fan. After treatment, the air is returned to the space via air outlet 14.

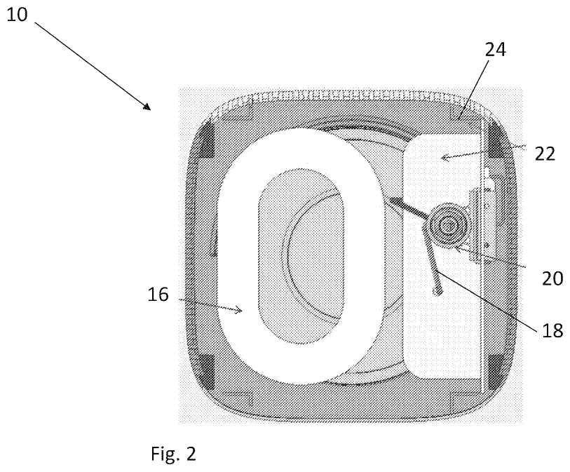

Reference is now made to FIG. 2, which shows a cutaway view of the device of FIGS. 1A to 1C as seen from above. Filter 16 filters the air sucked in from the air inlet 12. Dispenser 18 dispenses probiotics into the air once it has passed the filter. Dispenser 18 is part of a probiotic dispersion system that keeps and inserts probiotic material into the air. The probiotics are stored in bottle 20 which keeps the probiotic mixture alive for a defined period of time prior to being dispensed, and the bottle, along with filter 16, may be replaced at regular intervals. The probiotic dispersion system may be held within a box 22 which fits onto fitting 24 on the body of the device.

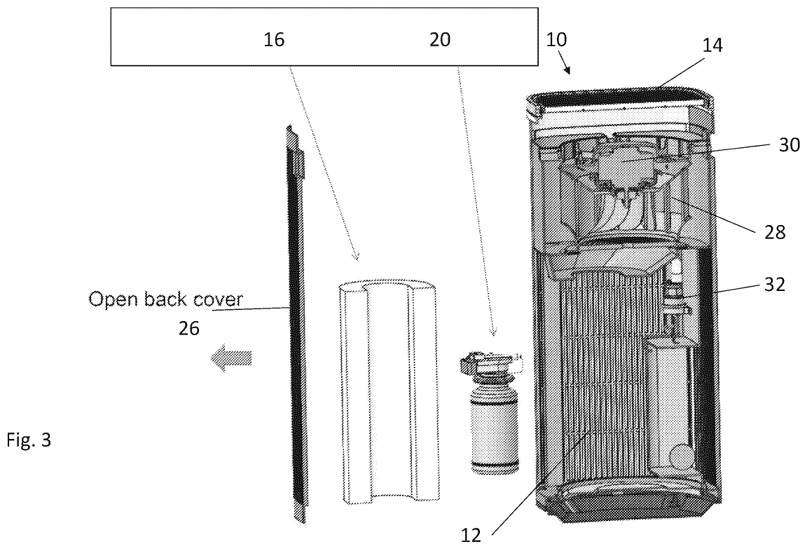

Reference is now made to FIG. 3, which illustrates a side cutaway view of the device 10 of FIGS. 1A-1C. Filter 16 is removed as is probiotics bottle 20 and removable back cover 26. Fan 28 may provide suction to move air through the filter and back out to via outlet 14.

Motor 30 may operate the fan, and a separate actuator 32 may operate the probiotic dispenser.

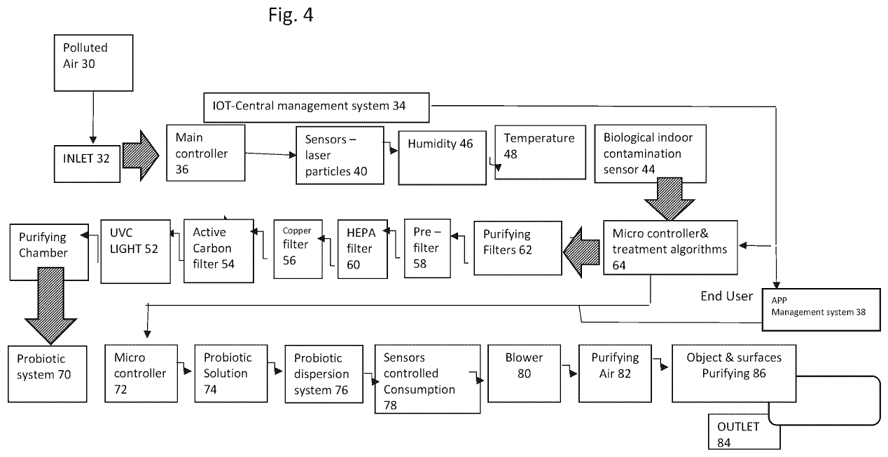

Reference is now made to FIG. 4, which is a simplified block diagram illustrating operation of the device of FIGS. 1A-1C.

Polluted air 30 from the internal space, say a room, a living space, a workspace, a factory space etc. is drawn in via inlet 32.

The device includes a central management system 34—which may be an IOT device, meaning it has connectivity to other devices and/or to the Internet in general, say via local wi-fi or hotspots or via a cellular connection. The central management system 34 operates a main controller 36 and may be connected to a user interface 38 which may be an end user management system. The end user is thus enabled to program and change settings on the device.

The device may include one or more sensors which may detect levels of air contamination in the internal space as well as environmental conditions. The sensors may include a particle sensor that counts particles in the air, for example a laser sensor. A biological sensor 44 may look for biological contamination. Environmental sensors may include a humidity sensor 46 and/or a temperature sensor 48.

Data from the sensors may be used by the microprocessor to vary the rate of air filtration. Thus for example the speed of fan motor 30 may be increased or decreased based on measurement results of the various sensors. Hence if the air is found to contain an increased number of particles the fan motor speed may be increased in order to filter more air over a given time. If the biological sensor finds more biological contamination then the dispensing of probiotics may be increased. In other words in the face of increased indoor biological contamination it is possible to increase the rate of insertion of probiotic material. Likewise it is possible to increase filtration levels. Accordingly infectious particles may be removed from the atmosphere before they can infect people using physical filtration and UV-C light, as will be discussed below.

In an embodiment, the main controller 36 stops filtering, or closes the filter during said insertion of probiotic material into the air. Accordingly, probiotic material is allowed to spread around the room and is not immediately lost in the filters following disposal.

The filter itself is part of a purifying chamber 50 and various technologies may be used, either separately or together to form an effective air filter. One of the technologies that may be used is UV-C light 52. UV-C light is a radiation method that makes use of a specific band of ultra-violet light to neutralize micro-organisms, for example by deactivating DNA, and thus disrupting the ability to multiply and cause disease. The UV-C light may thus removes micro-organisms from the air, allowing the micro-organisms in the probiotic material to replace them. A general problem with probiotics is that existing communities of flora do not provide space for new communities to establish themselves. The use of UV-C light to neutralize existing microorganisms, assisted by the other features of the filter, may allow the probiotic organisms a chance to establish themselves.

Another technology that may be used is an active carbon filter 54. The active carbon filter generally removes organic compounds from the air. A copper filter 56, may be provided as may a pre-filter 58 and high efficiency particulate filter HEPA 60. The various filters together work together to provide purifying filters 62. A separate microcontroller 64 may operate treatment algorithms using the filters and the probiotic system and based on the sensor measurements. It is noted that an H13 HEPA filter may filter out viruses including COVID 19.

The probiotic system 70 may include a separate microcontroller 72. The bottle 20 may contain a probiotic solution 74 which is intended to keep a probiotic mixture alive and effective for the period in between recharging the device. A probiotic dispersion system 76 serves to release small amounts of the probiotic solution into the filtered air under control of the treatment algorithms in microcontroller 64. That is to say the sensors and sensing measurements may control 78 the rate of consumption of the probiotic solution.

The refill bottle 20 and the filter are consumables. That is they get used up and have to be replaced, say every six months. The device may have an indicator light to indicate that the consumables are approaching expiry and need to be replaced.

A single fan or blower may be provided for the entire device, or a separate blower 80 may be provided to distribute the probiotic solution when the filter is switched off. In an alternative embodiment, a single blower is used and the filters are simply bypassed during distribution of the probiotic solution. The fan or blower may have a continuously variable speed or may have a specific number of set speeds, say four set speeds.

The purified air 82 proceeds to outlet 84 and the probiotic solution settles on objects and surfaces 86.

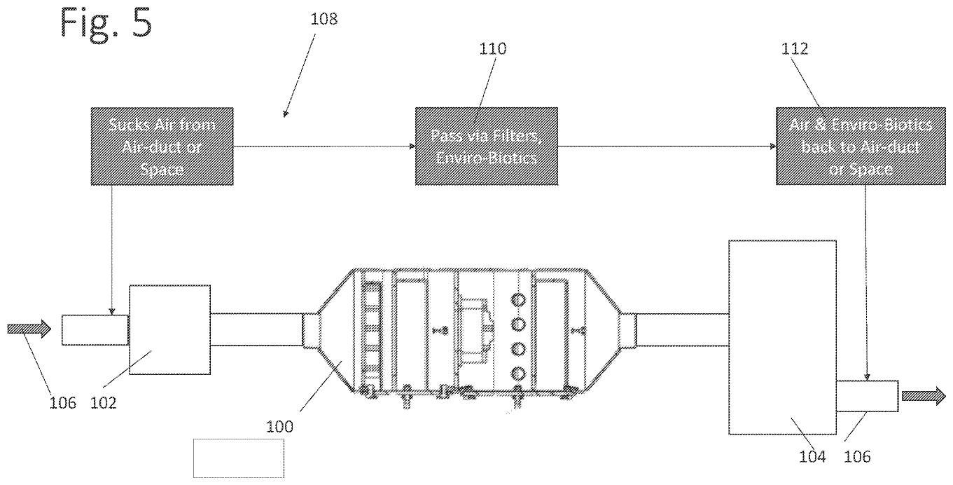

Reference is now made to FIG. 5, which is a simplified diagram illustrating an embodiment of the present invention which is integrated into heating, ventilation and air conditioning (HVAC) systems, typically of the kind found in commercial facilities. As shown in FIG. 5, an HVAC system 100 is connected via air ducts 102 and 104 to a facility. Air travels through the system in the direction of arrows 106. A bypass 108 according to the present embodiments sucks air from the air duct 102, passes the air via filters 110 of the kind referred to hereinabove and then inserts at unit 112, air enriched with environmental biotics. The enriched air is reinserted into duct 104.

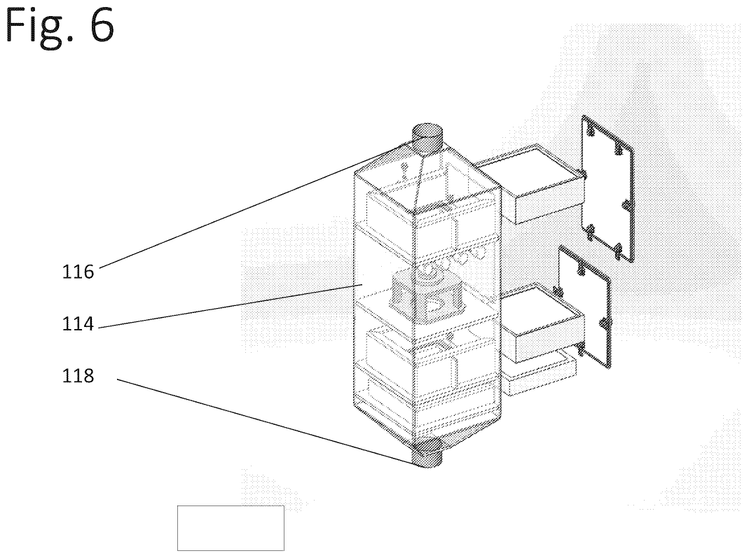

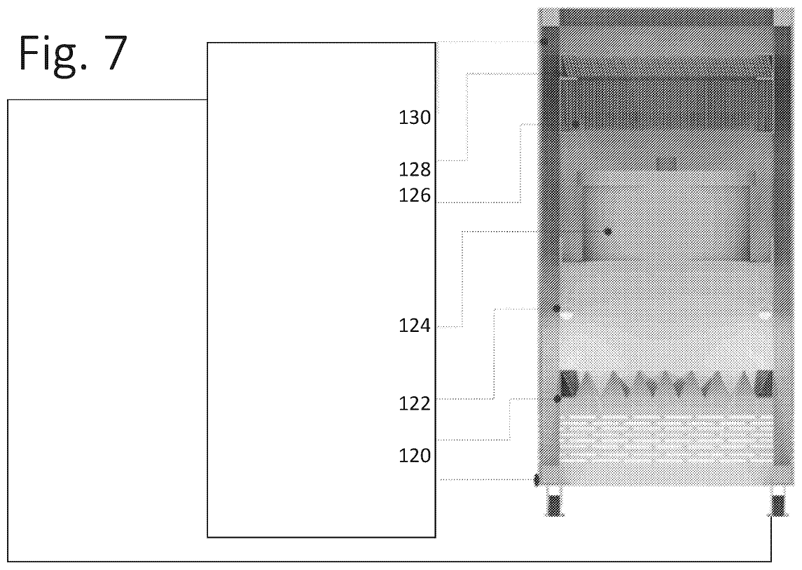

Reference is now made to FIG. 6, which is a simplified diagram illustrating a unit 114 for insertion around an air duct to provide a bypass as explained in FIG. 5. Inlet 116 receives bypass air from a duct that serves as an inlet to an HVAC system, and outlet 118 sends bypass air to inject to the outlet of the HVAC system. Within the unit 114 are filters, UV lights for sterilizing the air, and probiotic dispensers as well as bottles containing the probiotic material. A motor and fan may be included. Reference is now made to FIG. 7, which is a simplified diagram illustrating in greater detail the internal parts of a variation of the unit 114 of FIG. 7. The unit may be constructed of galvanized painted steel. A primary filter 120 carries out initial filtration of the incoming air. Ultra violet-C-lightbulbs 122 kill viruses. Blower 124 blows air through the system. ULPA final filtration filter 126 includes various other filters such as shown in FIG. 4 above. A charcoal filter 128 follows, which is the equivalent of the activated carbon filter 54 in FIG. 4. Insertion unit 130 inserts the probiotic materials.

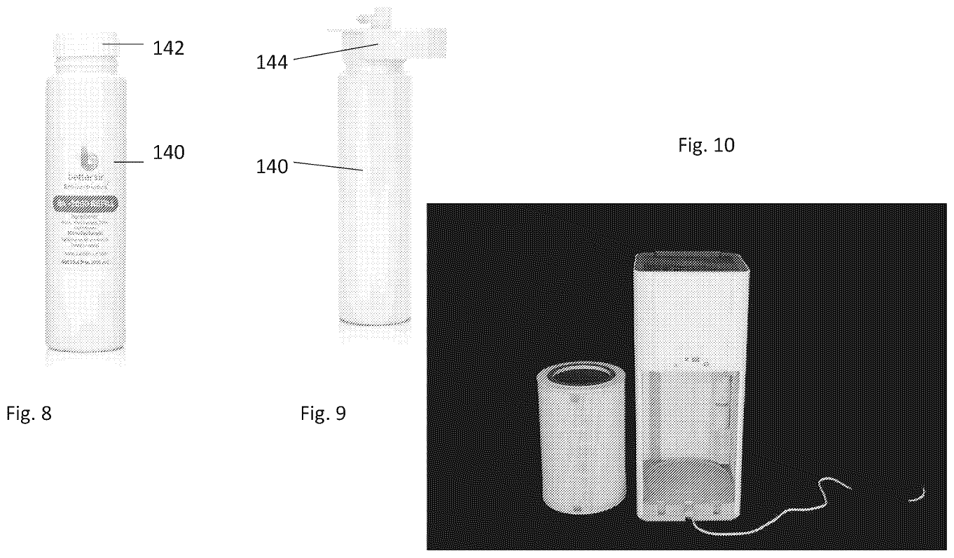

Reference is now made to FIG. 8 which illustrates a refill bottle 140 which contains the probiotic material. Prior to use the bottle is covered with removable cap 142.

Referring now to FIG. 9, the removable cap 142 is removed and dispensing head 144 is placed on the bottle 140. FIG. 10 illustrates the positioning of refill bottle 140 within the standalone dispensing unit of FIG. 1A. Positioning is similar within the unit 114 used with the HVAC systems.

As explained, the sensors within the unit allow regulation of the amount of probiotic material according to the state of the air in the space being treated.

It is expected that during the life of a patent maturing from this application many relevant filtering and probiotic technologies will be developed and the scopes of these and corresponding terms herein as well as other terms are intended to include all such new technologies a priori.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. Air purification device for internal spaces comprising:
an air inlet configured to receive air from an internal space;
a filter configured to filter air from said air inlet;
a probiotic dispersion system configured to insert probiotic material into said air after said filter; and
an outlet configured to distribute said air into said internal space, the device being configured to close said filter during said insertion of probiotic material.

2. The air purification device of claim 1, comprising at least one sensor to detect levels of air contamination in said internal space.

3. The air purification device of claim 2, wherein said at least one sensor comprises a particle sensor.

4. The air purification device of claim 2, wherein said at least one sensor comprises a biological sensor.

5. The air purification device of claim 2, wherein said at least one sensor comprises a humidity sensor and/or a temperature sensor.

6. The air purification device of claim 2, configured to vary a rate of air filtration based on measurement results of said at least one sensor.

7. The air purification device of claim 2, configured to vary a rate of said insertion of probiotic material based on measurement results of said at least one sensor.

8. (canceled)

9. The air purification device of claim 1, wherein said filter comprises a micron-scale filter.

10. The air purification device of claim 1, comprising an active carbon filter.

11. The air purification device of claim 1, comprising passing air through UV-C light.

12. The air purification device of claim 1, comprising a bypass configured to be positioned around heating, ventilation or air conditioning plant.

13. Air purification method for internal spaces comprising:
receiving air from an internal space;
filtering said received air;
inserting probiotic material into said air after said filtering;
distributing said air into said internal space; and
suspending said filtering during said inserting of probiotic material.

14. The air purification method of claim 13, comprising detecting levels of air contamination in said internal space.

15. The air purification method of claim 13, comprising detecting airborne particles.

16. The air purification method of claim 13, comprising detecting airborne biological contaminants.

17. The air purification method of claim 13, comprising detecting environmental conditions of said internal space.

18. The air purification method of claim 17, wherein said environmental conditions comprise one member of the group consisting of humidity and temperature.

19. The air purification method of claim 13, comprising varying a rate of air filtration based on measurement results of said air.

20. The air purification method of claim 13, comprising varying a rate of said insertion of probiotic material based on measurement results of said air.

21. (canceled)

22. The air purification method of claim 13, comprising one member of the group of filtering methods comprising:
micron-scale filtering; active carbon filtering and passing air through UV-C light.

23-24. (canceled)
